(19) United States
(12) Patent Application Publication
(10) Pub. No.: US 2010/0010615 A1
(43) Pub. Date: Jan. 14, 2010

(54) INSERTION SYSTEM FOR A MEDICAL DEVICE HAVING A SHEATH AND SHEATH FOR AN INSERTION SYSTEM FOR A MEDICAL DEVICE

(75) Inventors: Eugen Hofmann, Zurich (CH); Daniel Lootz, Rostock (DE)

Correspondence Address: BARNES & THORNBURG LLP, Suite 1150, 3343 Peachtree Road, N.E., Atlanta, GA, 30326-1428, US

(73) Assignee: BIOTRONIK VI PATENT AG, Baar (CH)

(21) Appl. No.: 12/495,927

(22) Filed: Jul. 1, 2009

(30) Foreign Application Priority Data
Jul. 8, 2008 (DE) 10 2008 040 252.4

(51) Int. Cl. A61F 2/84

(52) U.S. Cl. 623/1.11; 606/108

(57) ABSTRACT

An insertion system (100) for a medical device (10), which is situated in an outer sheath (20) upon insertion, the outer sheath (20) at least regionally encompassing an inner shaft. The outer sheath (20) at least regionally has, at least on its inner side (29) facing toward the medical device (10), a structure (22, 32) which is complementary with an external-peripheral segmented structure (12) of the medical device (10) like a bolt-nut connection.

### PRIORITY CLAIM

This patent application claims priority to German Patent Application No. 10 2008 040 252.4, filed Jul. 8, 2008, the disclosure of which is incorporated herein by reference in its entirety.

### FIELD

The present disclosure relates to an insertion system for a medical device having a sheath. The present disclosure also relates to a sheath for an insertion system for a medical device.

### BACKGROUND

The use of catheters for the release of medical devices such as stents or dilation balloons is known. One area of application relates, for example, to angioplasty in which balloon catheters are inserted into blood vessels, pushed up to a stenosis, and expanded there in order to remove the constriction of the blood vessel. A guide wire having a small diameter typically projects beyond the balloon catheter on the distal end.

The insertion of the medical device is performed using an insertion system, a so-called stent delivery system. Thus, an insertion system for a stent is known from European Patent Application No. 1 601 312, for example, in which the stent is situated on a rotatable sheath for better positioning of the stent. Forks in the blood vessel can be better navigated upon insertion of the stent therewith.

However, there are specific stent structures which may only be released with difficulty using the known insertion systems, for example, for self-expanding stents. The release occurs in that an outer sheath, which encloses the stent upon insertion, is retracted. Above all, stent structures having little cross-linking and, in particular, stents whose spacing to adjacent elements is great tend to bunch up and block when the stents are to be released. Retraction of the outer sheath is made difficult or even prevented.

### SUMMARY

The present disclosure describes several exemplary embodiments of the present invention.

One aspect of the present disclosure provides an insertion system for a medical device which is situated in an outer sheath upon insertion, comprising a) an outer sheath having an inner side, wherein the outer sheath has a structure which is complementary to an external-peripheral segmented structure of the medical device, wherein the structure is disposed at least partially on the inner side of the outer sheath which is complementary to an external-peripheral segmented structure of the medical device; and, b) an inner shaft at least partially encompassed by the outer sheath.

Another aspect of the present disclosure provides an outer sheath for an insertion system for a medical device, comprising an outer sheath having an inner side, wherein the outer sheath has a structure which is complementary to an external-peripheral segmented structure of the medical device, wherein the structure is disposed at least partially on the inner side of the outer sheath which is complementary to an external-peripheral segmented structure of the medical device; and, an inner shaft at least partially encompassed by the outer sheath, wherein a threaded structure is disposed at least on the inner side, which allows the execution of a rotational movement around a complementarily implemented structure in the operating state.

The present disclosure provides an insertion system for a medical device in which the release of the medical device is made easier, even if the stent structure tends to bunch up.

The present disclosure also provides an insertion system for a medical device which is situated in an outer sheath upon insertion, in particular, an insertion system for a self-expanding stent, the outer sheath at least regionally enclosing an inner shaft. The outer sheath is at least regionally has, at least on its inner side facing toward the medical device, a structure which is complementary to an external-peripheral segmented structure of the medical device like a bolt-nut connection. The inner shaft carries the stent in the distal area while the outer sheath holds the stent in the compressed, crimped state. The outer sheath may advantageously be retracted by rotating, even with stents in which the predominant number of the webs of the radial support structure are situated at an angle to the axial direction of the stent and thus to the outer sheath.

Upon release of the medical device, the complementary structure of the outer sheath may preferably be positively guided on the segmented structure in a rotational movement. In the normal case, the stent is released by the retraction of the outer sheath. In this case, the outer sheath may be retracted while rotating. Axial compression forces which act on the stent may thus favorably be minimized. The rotational movement of the outer sheath may preferably run along a spiral-shaped meandering structure of a stent.

The outer sheath may preferably be torsionally rigid in order to make a rotational movement easier.

The distance which the outer sheath is movable per revolution in the axial direction may expediently be adapted to a pitch of the segmented structure.

If the outer sheath of the insertion system may have an external thread at least on its proximal end, using which a rotational movement in relation to the medical device is activatable, the release may be caused in that, for example, a rotary crank or a suitable other device is actuated, which may act on the external thread and separates the outer sheath from the medical device to be released by rotation.

Even with axially long medical devices, such as stents, a release of this type may be performed without problems using the preferred insertion system, if the outer sheath may comprise a low-friction polymer. For example, fluoropolymers, such as TEFLON®, are preferable. The friction between the segmented structure of the medical device and the interior of the outer sheath may advantageously be reduced.

The outer sheath of the insertion system may preferably have an internal thread structure on the inner side as a complementary structure to the segmented structure of the medical device.

To increase its torsional rigidity, the outer sheath of the insertion system may favorably have a reinforcement. Thus, a braid made of one or more round or flat wires may be situated as a reinforcement on a hose. The reinforcement may also be embedded in the hose.

The outer sheath of the insertion system may comprise a tube which has cuts having a pitch. The cuts are used as a thread having a pitch.

The insertion system may advantageously have a blocking device in order to prevent twisting of the medical device during the release. The inner shaft may thus be designed, at least in the proximal area of the carrier of the medical device, so that the medical device is fixed. A blocking device of this type may also be provided between the proximal and distal ends of the insertion device.

The outer sheath of the insertion system may have a structure similar to a thread at least on the inner side which allows the execution of a rotational movement around a complementarily implemented structure in the operating state.

The structure may advantageously be implemented as a sliding track or notch on the inner side. The sliding track may be applied as a coating, preferably as a metallic coating, or may be implemented as a hardened area on the inner side. Such hardening may be performed by a suitable surface treatment, for example, hardening by thermal treatment and/or by irradiation using particles or the like.

To improve the torsional rigidity of the outer sheath, a reinforcement may expediently be provided.

An external thread may preferably be provided on one end of the outer sheath for activating the rotational movement of the outer sheath.

The outer sheath may comprise a tube with cuts having a pitch.

Furthermore, the present disclosure provides an outer sheath for an insertion system in which a structure similar to a thread is implemented at least on the inner side which allows the execution of a rotational movement around a complementarily implemented structure in the operating state.

The structure may advantageously be implemented as a sliding track or notch on the inner side and/or be applied as a coating. The sliding track may be implemented as a hardened area on the inner side.

If the body of the outer sheath is formed from a hose, a reinforcement may expediently be provided to improve the torsional rigidity.

The outer sheath may comprise a tube which has cuts having a pitch. The cuts may be provided in their design with interruptions, their pitch, and their spacing so that the torsional rigidity of the outer sheath is adequately ensured.

An external thread may preferably be provided on one end of the outer sheath for activating the rotational movement of the outer sheath.

### BRIEF DESCRIPTION OF THE DRAWINGS

Various aspects of the present disclosure are described hereinbelow with reference to the accompanying figures. The invention is explained in greater detail hereafter for exemplary purposes on the basis of exemplary embodiments shown in drawings.

FIG. 1 shows a schematic view of a section through one exemplary embodiment of an insertion system;

FIG. 2 shows a stent having a segmented structure which is susceptible to bunching;

FIG. 3a shows a first exemplary embodiment of a reinforced outer sheath in an external view;

FIG. 3b shows a second exemplary embodiment of a reinforced outer sheath in an external view;

FIG. 4 shows an internal view of an outer sheath having internal thread;

FIG. 5a shows a perspective view of an exemplary embodiment of an outer sheath in overview, the outer sheath having a spiral-shaped laser cut which significantly increases the flexibility of the tube; and

FIG. 5b shows a perspective view of an exemplary embodiment of an outer sheath in detail, the outer sheath having a spiral-shaped laser cut which significantly increases the flexibility of the tube.

Functionally identical or identically-acting elements are each numbered using the same reference numerals in the figures. The figures are schematic illustrations of the present invention. The figures image nonspecific parameters of the present invention. Furthermore, the figures only illustrate typical designs of the present invention and are not to restrict the present invention to the designs shown.

### DETAILED DESCRIPTION

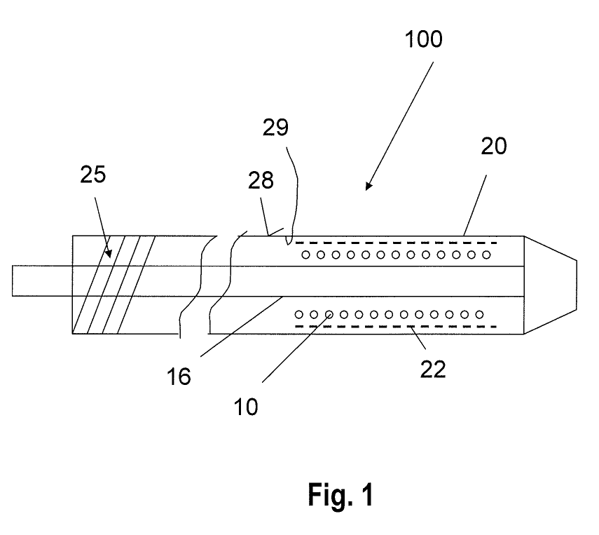

FIG. 1 schematically shows one exemplary embodiment of an insertion system 100 for a medical device 10, which is implemented as a self-expanding stent. In the mounted state for insertion into a vessel or organ, the medical device 10 is mounted on the distal end of an inner shaft 16 and encompassed by an outer sheath 20 which at least regionally encloses the inner shaft 16. The outer sheath 20 is retracted to release the stent. The outer sheath 20 has a structure 22 on its inner side 29 which works together with an external-peripheral structure of the medical device 10 so that, upon release, a rotational movement of the outer sheath 20 may occur in relation to the medical device 10 so that axial compression may be avoided.

The outer sheath 20 has an external thread 25 on its proximal end which may be used to activate a rotational movement to release the medical device. One or more blocking devices (not shown) are expediently provided which may prevent undesired twisting upon insertion of the medical device 10 to its usage location.

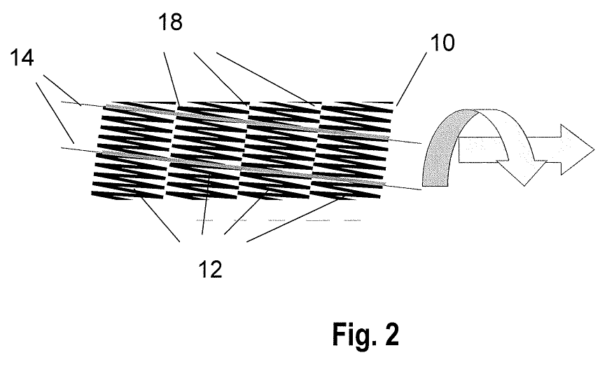

The medical device 10, which is implemented as a stent, may have a segmented structure 12 as shown in FIG. 2. The segmented structure 12 comprises a meandering structure oriented in a spiral (strut elements). The boundaries 18 of the segmented structure 12 predetermine a pitch like a thread. If the outer sheath 20 is retracted axially without rotational movement, the retraction may result in bunching of the segmented structures. According to one exemplary embodiment of the present disclosure, the outer sheath 20 may be rotated like a nut on a bolt, the rotational movement also causing an axial displacement of the outer sheath 20. This rotating-lifting movement is indicated by the arrow to the right in FIG. 2.

The outer sheath 20 (FIG. 1) preferably at least regionally has, at least on its inner side 29 facing toward the medical device 10, a structure 22 which is complementary with the external-peripheral segmented structure 12 of the medical device 10 like a bolt-nut connection.

The outer sheath 20 can be positively guided in a rotational movement by the pitch of the segmented structure 12. The distance by which the outer sheath 20 is movable per revolution in the axial direction is adapted to the pitch of the segmented structure.

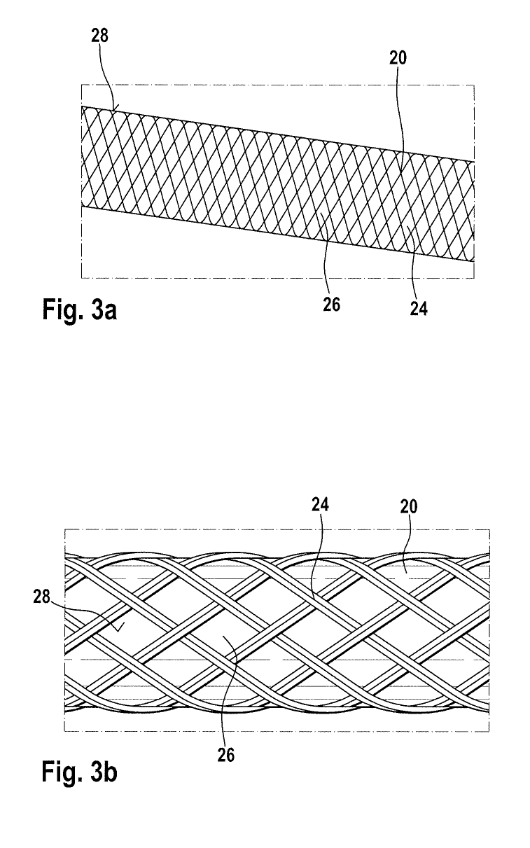

The outer sheath 20 is expediently implemented as torsionally-rigid. FIGS. 3a and 3b show examples of a torsionally-rigid design of an outer sheath 20. The body 26 of the outer sheath 20 is formed by a hose made of a polymer having good friction properties, such as a fluoropolymer, for example, around which a braid made of one or more round or flat wires is applied as a reinforcement or is embedded in the hose.

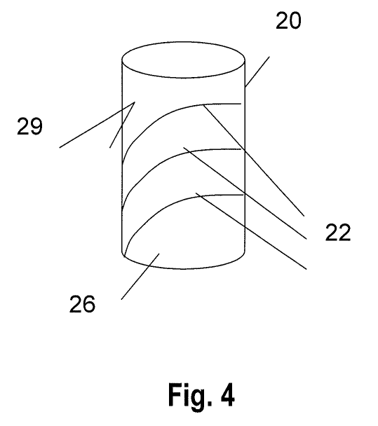

FIG. 4 shows an exemplary embodiment of an internal view of an outer sheath 20 having a polymer body 26 in which a complementary structure 22 is incorporated as an internal thread. The complementary structure 22 may be implemented as a sliding track or notch on the inner side 29 which may be implemented, for example, as a metallic coating or as a hardened area on the inner side 29.

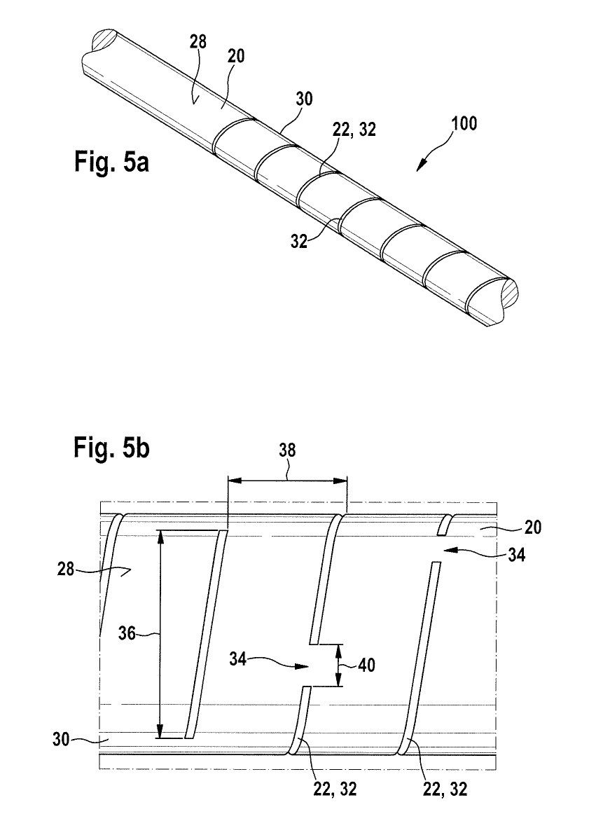

FIGS. 5a and 5b show an exemplary embodiment of an outer sheath 20 which is formed from a body 30 in which cuts 32 are incorporated as the complementary structure 22. The rigidity of the outer sheath may be influenced by the differing exemplary embodiments of the cuts 32 in the outer sheath 20. The length 36 of the cuts 32, their axial spacing 38, and one or more interruptions 40, realized by uncut sections 34 may be intentionally set so that a sufficient torsional rigidity results. The outer sheath 20 is preferably made of metal. In FIGS. 5 and 6, the desired flexibility may be set depending on the pitch.

Using the insertion system 100 and the outer sheath 20 as disclosed hereinabove, medical devices 10, in particular, stent structures, may advantageously be released which could not be released or could only be released with difficulty using known insertion systems. In particular, a reduction of the friction resistance and the release force connected thereto may be achieved. The advantage results for the design of stent structures is that stent structures may be designed as more flexible or less sensitive to fatigue fractures.

All patents, patent applications and publications referred to herein are incorporated by reference in their entirety.

What is claimed is:

1. An insertion system for a medical device which is situated in an outer sheath upon insertion, comprising:
a) an outer sheath having an inner side, wherein the outer sheath has a structure which is complementary to an external-peripheral segmented structure of the medical device, wherein the structure is disposed at least partially on the inner side of the outer sheath which is complementary to an external-peripheral segmented structure of the medical device; and,
b) an inner shaft at least partially encompassed by the outer sheath.

2. The insertion system of claim 1, wherein upon the release of the medical device, the complementary structure of the outer sheath can be positively guided in a rotational movement on the segmented structure.

3. The insertion system of claim 1 wherein the outer sheath is implemented as torsionally-rigid.

4. The insertion system of claim 1, wherein the distance which the outer sheath is movable per revolution in the axial direction is adapted to a pitch of the segmented structure.

5. The insertion system of claim 1, wherein the outer sheath has an external thread at least on its proximal end, using which a rotational movement in relation to the medical device is activatable.

6. The insertion system of claim 1, wherein the outer sheath comprises a low-friction polymer.

7. The insertion system of claim 1, wherein the outer sheath has an internal thread structure on the inner side as the complementary structure.

8. The insertion system of claim 1, wherein the outer sheath has a reinforcement to increase its torsional rigidity.

9. The insertion system of claim 1, wherein the outer sheath comprises a tube which has cuts having a pitch.

10. An outer sheath for an insertion system for a medical device, comprising: an outer sheath having an inner side, wherein the outer sheath has a structure which is complementary to an external-peripheral segmented structure of the medical device, wherein the structure is disposed at least partially on the inner side of the outer sheath which is complementary to an external-peripheral segmented structure of the medical device; and, an inner shaft at least partially encompassed by the outer sheath, wherein a threaded structure is disposed at least on the inner side, which allows the execution of a rotational movement around a complementarily implemented structure in the operating state.

11. The outer sheath of claim 10, wherein the structure is a sliding track or notch on the inner side.

12. The outer sheath of claim 11, wherein the sliding track is applied as a coating.

13. The outer sheath of claim 11, wherein the sliding track is a hardened area on the inner side.

14. The outer sheath of claim 10, further comprising a reinforcement to improve the torsional rigidity.

15. The outer sheath of claim 10, wherein an external thread is provided on one end.

16. The insertion system of claim 10, wherein the structure is a bolt-nut connection.
